(19) United States
(12) Patent Application Publication
(10) Pub. No.: US 2003/0019150 A1
(43) Pub. Date: Jan. 30, 2003

(54) Reclamation system for agricultural runoff

(76) Inventors: Benedict B. St.Onge, San Marco, CA (US); Jerry Smith, Fallbrook, CA (US)

Correspondence Address: KOPPEL & JACOBS, AN ASSOCIATION OF PROFESSIONAL, LAW CORPORATIONS, 555 ST. CHARLES DRIVE, SUITE 107, THOUSAND OAKS, CA, 91360, US

(21) Appl. No.: 09/844,215

(22) Filed: Apr. 27, 2001

(51) Int. Cl. A01B 79/02; A01C 1/00; A01G 1/00; A01H 3/00; A01B 79/00

(52) U.S. Cl. 47/1.01 R

(57) ABSTRACT

The invention relates to systems for collecting excess water applied to crops, treating the collected water and reusing the treated water for agricultural purposes or delivering it to ground water streams with so as to achieve cost savings, healthier plants, and reduce the environmental burden. The cost of providing water to crops, the amount of herbicides and/or pesticides delivered to the crops and the quantities of nitrogen fertilizer required is significantly reduced by a) capturing as much as possible of the water provided to the planting area which is not taken up by the planted crops, b) treating that captured water with ozone and c) reapplying the ozone treated water to the crops. The quality and quantity of food stuffs produced is also increased by the process.

The invention relates to the reclamation of water runoff from crop irrigation with the subsequent reuse of the reclaimed water. The runoff water is collected, and treated with ozone to eliminate or significantly reduce pesticides, herbicides or other organic material in the water that may be detrimental to underground water reserves or surface water streams if allowed to escape untreated. This procedure allows valuable inorganic fertilizers or desirable minerals previously applied to the plants or leached from the soil to be returned to the plants, thus replenishing plant nutrients without requiring additional purchased plant feeds.

### BACKGROUND

Many pesticides or herbicides are toxic to humans and animals. Considerable environmental and ecological damage can occur when these compounds are removed from the vicinity of plants to which they are applied by excesswater that does not enter the soil (ie runoff water) or they are carried to subterranean water by irrigation water applied to the cultivated fields. This water can also leach desired minerals and nutrients from the soil as it runs off from the planting beds. It is desirable to minimize the leaching of these desirable material or, if that material is leached from the soil, to return it to the planting beds.

For example, it is reported that plant nurseries have in excess of 24,000 acres container grown plants. (USDA, Horticulture Specialties, 1991) These nurseries will apply from 1036 to 4730 pounds of nitrogen based fertilizer to an acre of potted plants. These nursery operations, with an application rate of 1,500 pounds/acre, will use about 37 million pounds of nitrogen based fertilizer a year (EPA) Only about 6% is retained by the plants resulting in about 34 million pounds of fertilizer being leached into the environment from these nursing operations. According to the USDA, there were over 332 million acres of farm land planted with crops in 1997 in the US. While considerably less nitrogen fertilizer is applied on a per acre basis to in-ground crops, 11.4 million tons of nitrogen fertilizer, at about $1.00 per pound, was sold in 1992. The result is that an enormous amount of the nitrogen based fertilizer applied to crops leaches into the environment each year, largely due to water runoff and seepage into the soil. It has been estimated that a typical sprinkler irrigation system generates about 100,000 liters (26,400 gallons) of nutrient rich waste water per hectare (2.471 acres) per day (The Nursery Papers, Nursery Industry Association of Australia). According to one source, the toxic release in US surface water discharges in 1998 was in excess of 3.8 million pounds nitrates comprising 2.4 million pounds. An estimated 60% of river pollution comes from agricultural runoff. As of 1998, 3.8 million acres of lakes and reservoirs and 84,000 miles of rivers and streams in the US contain nutrients from runoff to the extend that they can no longer support aquatic life.

It is also desirable to assure, to the greatest extent possible, that pesticides and herbicides are applied to the plants and planting beds in known and controlled quantities so as to obtain the desired effect on the plants. However, when these materials wash from the plant area by irrigation water or storm runoff, care must be taken that they are not allowed to seep into the underground water and contaminate drinking water which is drawn from local wells or that run off into surface water streams, which harms fish and local wild life Additionally, contamination of feed streams, which eventually become our source of drinking water, is not desirable. The Clean Water Act embodies the principal concept that all discharges into the nation's waters are unlawful unless specifically authorized by permit. As part of the permitting process, the EPA, state and local water control agencies have been promulgating numerous regulations which require that the runoff water be severely curtailed and that the water that is allowed to escape from planting fields meet certain quality standards. These standards can not be met by many agricultural operations.

A still further problem is an increasing limitation on the clean water available for crop irrigation, and the subsequent increase in the cost of the water used. This is a further incentive to assure that a significant amount of the water applied to the crops is consumed by the crops, seepage of water into the soil is limited to that which is necessary to nourish the roots of the crop and excess water is not lost to underground water reserves. It is further desirable that the water that does not seep into the soil and runs off is captured and reused to the greatest amount possible and that the water reclaimed and recycled is cleaned of undesirable organic contaminants so that the farmer has close control of the quantity of pesticides and herbicides applied to the crops.

Therefore, there is a need for economical and efficient means for collecting, treating and returning agricultural water to crop irrigation systems and assuring that the water reused for irrigation is not detrimental to the plant's health.

Ozone has been used as an oxidizing agent for the treatment of biological materials in municipal sewage treatment plants for the production of potable water. In the agricultural environment, it is also known that ozone can be beneficial in treating agricultural animal wastewater. Pollution problems from manure and liquid animal waste in farm animal confinement areas, such as barns and farm yards used for swine, poultry or dairy cow include nitrogen, phosphorus, solids and bacteria. They emit foul odors as well as generate ammonia, methane and hydrogen sulfide from waste collection areas, such as collection ponds. In addition, these ponds tend to leak into under ground water sources as well as overflow, contaminating local ground water. These operations typically use anaerobic digestion ponds. Alternatively, U.S. Pat. No. 6,193,889 issued Feb. 27, 2001, describes a system employing closed tanks for a process utilizing solids separation, nitrification/denitrification and precipitation procedures. The clarified wastewater is then vigorously aerated to stimulate nitrification, followed by denitrification. The final step is sterilization using ozone.

Ozone has also been injected directly into planting soil as part of the planting process. Wickramanayayake (U.S. Pat. No. 5,269,943) teaches that the long-term treatment of soils with unstabilized ozone dissolved in water is not feasible because the ozone decomposes to rapidly. However, Pryor, U.S. Pat. No. 6,173,527, describes the gaseous deliver of ozone to soil which has been saturated with water to sanitize the soil and kill organisms in the soil. Gaseous ozone applied to soil prior to or at the time of planting promotes the growth of various crops planted in the treated soil. Pryor (U.S. Pat. No. 5,566,627) also describes the delivery of gaseous ozone to soil during tilling, fertilization, irrigation, sowing seeds or transplanting plants, spraying herbicides or pesticides, and harvesting crops. The gaseous ozone is delivered to relatively dry soil as a replacement for the use of methyl bromide or other fumigating biocides to kill undesirable living organisms such as micro-organisms, multicellular animals, plants and seeds.

Augustine et. al (U.S. Pat. Nos. 5,194,147 and 5,078,88) provides a method and system for the decontamination of wastewater from greenhouse runoff which utilizes a collection system incorporating a holding tank where the wastewater is aerated to promote degradation of toxic materials (pesticides, herbicides, fumigants, carriers, wetting agents, adjuvants, etc.). The aerated water from the holding tank is then distributed over and passed through a soil bed reactor where microbial action further degrades toxic materials. The discharge from the soil bed reactor is then feed to a pond stocked with plant and animal species which promote further degradation of toxins. A conventional ozonization unit may be employed to add ozone to the water in the holding tank.

It has also been proposed to treat edible plants with water containing carbon dioxide and ozone to improve growth, yield and quality of the plant and foodstuffs obtained therefrom. (U.S. Pat. No. 5,561,944 to Ismail et al.). CO₂ and ozone are added to the water immediately prior to the water being applied to the plants. This process is said to reduce microbial activity in the soil and in the roots of the plants, improve the texture, shape, color, and taste of treated fruits and vegetables and increase the fiber, sugar and carbohydrate content of the food stuff produced.

Persinger, U.S. Pat. No. 5,697,187, adds gaseous ozone directly to a stream of irrigation water immediately before it is applied to crops. This was said to improve crop quality by stopping surface fungus and molds on the above ground portion of the crops. The process increased the water penetration of the soil, reduced the water requirements by one-half and reduced the need for fungicides.

Likewise, Smith et al, U.S. Pat. No. 5,816,498, adds ozone to irrigation water immediately prior to application to the crops. The ozone generator was mounted on a tractor and the ozone was injected into water as it was applied to the crops from a tractor traversing the planted field. Use of water with a high level of ozone sprayed directly on crops was found to prevent various bacterial and fungal disease on the crops.

While ozone has been injected into soil prior to and during the growing period, water with dissolved ozone has been applied directly to crops to improve crop quality, and ozone has been used as part of waste water and sewage treatment process, no one has shown or suggested the collection of excess (runoff) water following irrigation of crops in combination with the treatment of that runoff water with ozone followed by the reapplication of that treated water to the crops. As an added advantage, the collection of that runoff water significantly reduces the amount of contaminated water, which passes through the soil, carrying with it, nutrients in the soil, into the subterranean water table or flows into surface water streams.

### SUMMARY

It has now been found that the cost of providing water to crops, the amount of herbicides and/or pesticides delivered to the crops and the quantities of nitrogen fertilizer required can be significantly reduced by a) capturing as much as possible of the water provided to the planting area which is not required by the planted crops, b) treating that captured water with ozone and c) reapplying the ozone treated water to the crops. It has also been found that the quality and quantity of food stuffs produced can be increased by this process. The invention relates to systems for collecting excess water applied to crops, treating the collected water and reusing the treated water for agricultural purposes or delivering it to ground water streams with so as to achieve cost savings, healthier plants, and reduce the environmental burden.

### DRAWINGS

These and other features, aspects and advantages of the present invention will become better understood with reference to the following description, appended claims and accompanying drawings where:

FIG. 1 is a schematic block diagram of a collection and treatment system incorporating features of the invention.

FIG. 2 is a graph showing projected water costs.

FIG. 3 is a graph showing projected cost savings of water due to reclamation and recycling of agricultural water.

FIG. 4 is a graph showing the average cost of nitrates used for fertilizers.

FIG. 5 is a graph showing projected cost savings of nitrate fertilizer due to reclamation and recycling of agricultural water.

FIG. 6 is a schematic block diagram of a preferred treatment system.

### DETAILED DESCRIPTION

Water that runs off from crops is routed to a collection system where it is treated with ozone to significantly reduce the quantity of undesirable organic materials, such as pesticides, herbicides, pathogens, etc. This process may be enhanced by also exposing the collected water to ultraviolet light and/or activated charcoal to remove additional hydrocarbons and other adsorbable organic materials.

Many nurseries and farms are making efforts to collect all excess water by applying plastic covers to soil surrounding crops to keep down weed growth, restrict applied water primarily to the plant base and its root: system, and to direct any runoff to drainage troughs and piping systems which may be constructed of plastic pipe or sheeting. Nurseries typically recover 50% of the water applied to their plantings, farms recover about 40% of the water applied to crops. There is also an incentive to bury plastic liners in the soil sufficiently below the soil surface so as to not disrupt the root system of the plants. These plastic liners, in conjunction with underground plastic lined drainage systems, provide a means to collect and recycle water that seeps beyond the plant root system. Water, and the nitrates and other minerals carried by that water, will typically travel about 1 meter a day through soil to which it is applied. Under laying plastic liners can also provide a significant benefit in recovering and recycling water, and dissolved plant nutrients applied to park areas and golf courses. Therefore, in a matter of hours after application, water, and the fertilizer in it, may no longer be available to the plants through its roots system. The collected surface and subterranean water can then be collected in ponds and tanks for treatment and discharge or reuse. However, current treatment processes are inadequate or expensive for the production of reusable water and generally remove nitrates from the water. It has been found that the removal of nitrates and other minerals is unnecessary and undesirable if the water is to be returned to the planting area, The only processing necessary is to treat the water with ozone and possibly expose the ozonated water to UV light to remove undesirable organic materials.

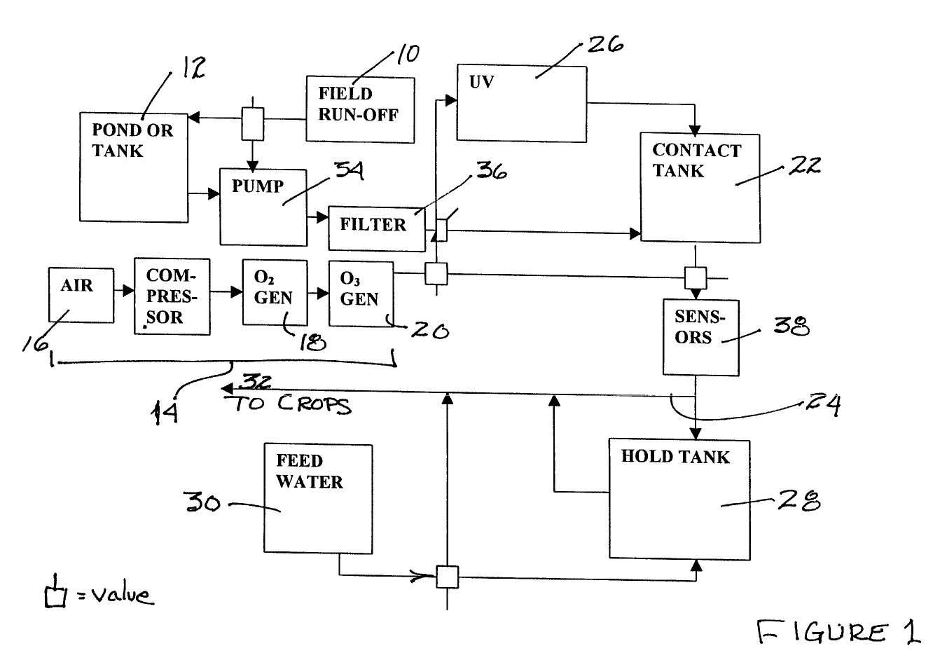

Referring to FIG. 1, a water reclamation system incorporating various alternative features of the invention is shown. Water collected from crop runoff 10 is stored in holding ponds or tanks 12 for subsequent treatment. Alternatively, in one version of a continuous processing system as shown in FIG. 6, the runoff water is feed directly through the treatment equipment followed by storage for later delivery or returned directly to the irrigation system for reapplication to the crops.

The runoff water is pumped through a piping system where it is mixed with ozone provided by an ozone generation system 14. The ozone generation system typically comprises a compressed air supply 16, oxygen enriched air 18 or an oxygen supply that is feed through an ozone generator 20. A preferred ozone generator is a high voltage unit such as shown in U.S. patent application Ser. No. 09/793,795. However, any ozone generator capable of providing a sufficient quantity of ozone is acceptable. The preferred system should be able to provide 1.5 to 5.0 pounds of ozone to about 100,000 gals of water being treated on a daily basis. This is based on metals and organics analysis in typical run off water analysis reports. However, one skilled in the art will recognize that the amount of ozone necessary will depend on the quantity of organic chemicals in the water stream being treated.

The ozonated water can then be fed to a contact tank 22. This is sized to provide a predetermined delay or dwell time for the runoff water to allow the Ozone sufficient time to react with the organic contained in the water. Alternatively, the water can be fed directly back into the irrigation system 24. As an alternative, the ozonated water can also be passed through piping or tanks 26 where the water is expose to ultraviolet (UV) light in the 100-280 nm frequency range, preferably 254 nm. 260 watts power input is delivered to the UV bulbs so that the water being treated is exposed to at least about 100 watts of output power. It has been found that exposing ozonated water to UV light results in increased efficacy in destroying bacteria. This combination is particular effective where there is a heavy bacterial load in the water. Once the bacteria is reduced by the UV/ozone treatment a second addition of ozone may be used to reduce pesticides which may not have been adequately destroyed because the early injection of ozone was depleted by the high bacterial content. The treated water can then be stored in holding tanks 28 or, mixed with fresh feed water 30, applied directly back to the same crops 32 or to other crops in need of water and/or fertilizer.

It should be recognized the additional plumbing components normally included in a water handling system may be included. These include, but are not limited to, pumps 34 to increase the pressure of the water delivered, check valves (not shown) to assure that the water flows in the desired direction, filters 36 to remove solid matter or particulates introduced with the feed stream or generated during the processing of the water, various sensor 38 to monitor water quality, bypass and storage tanks in addition to those shown in FIG. 1. In addition, carbon filters can be added to the system to adsorb or absorb some of the organic materials in the feed steam. If carbon filters 36 are placed in the system before the feed stream is exposed to the ozone, the contaminant load that must be reduced by the ozone is reduced, thus making the ozone treatment more efficient and effective in lowering the concentration of pesticides, herbicides, fungicides, etc.

Because inorganic compounds such as nitrates and phosphates dissolved in the water or applied for fertilization purposes, as well as desirable minerals in plant food compositions or leached from the soil, are not effected by the ozone and/or UV treatment they are reclaimed with the water and subsequently fed back to the crops. While the savings depend on the amount of fertilizer and water applied and the type of soil the crops are planted in, up to about 6000 pounds of nitrates can be saved and recycled per an acre-foot of water reclaimed by this process. This results in a significant cost saving for fertilizer and a significant reduction in contamination of surrounding ground water as a result of the recapture of the nitrates not initially utilized by the crops which normally find there way into the ground water.

It has also been found that the redelivery of the ozone treated water to crops reduces the amount of pesticides required to produce quality produce. While ozone has a half-life of about 20 minutes when dissolved in water, about 25% is still present after 40 minutes and about 12% is still present after 60 minutes. Taking into account some further degradation of the ozone due to reaction at the contact surfaces of the piping, tanks and other components in the system, if the ozone treated water is delivered to the crops within one hour of treatment, there is apparently sufficient ozone remaining in the water to destroy mold, bacteria, fungus, certain insects and insect eggs and other pathogens in the soil or on the plants which normally effect the quality of the plants, thus decontaminating the plant surface and surroundings without negatively effecting the plant itself.

### EXAMPLE 1

A system such as shown in FIG. 1 was used to treat water collected in a collection pond from a plant nursery. Approximately 3.2 pounds per of ozone was generated and feed uniformly over a 24 hour period into a continuously flowing steam of water drawn off from the pond at a rate of about 75 gallons per minute (108,000 gallons/24 hours). The treated water was collected in a separate holding tank. UV treatment and carbon filters were used. Table 1 lists the water analysis of the pond water fed to the system and the treated water sample within 4 hours after it was collected in the holding tank.

TABLE 1
NURSERY WATER ANALYSIS

| | Pretreat | Post-treat | % change |
|---|---|---|---|
| Calcium, ppm | 76.5 | 65.1 | −13.5% |
| Copper, ppm | 0.049 | 0.023 | −53.1% |
| Iron, ppm | 1.20 | 1.11 | −07.5% |
| Magnesium, ppm | 28.5 | 24.9 | −09.4% |
| Manganese, ppm | 0.05 | 0.02 | −60.0% |
| Potassium, ppm | 40.1 | 20.5 | −48.4% |
| Sodium, ppm | 90.3 | 79.7 | −11.7% |
| Zinc, ppm | 0.034 | 0.057 | +67.6% |
| Bicarbonate, ppm | 110.0 | 122.0 | +10.7% |
| Total Alkalinity as | 90.0 | 100.0 | +10.0% |
| CaCo3, ppm | | | |
| Total Hardness, ppm | 308.0 | 265.0 | +14.0% |
| Specific Conductance, | 1090.0 | 823.0 | −24.5% |
| ppm | | | |
| Chlorine, ppm | 92.9 | 82.7 | −11.0% |
| Nitrate (as NO3), ppm | 131.0 | 56.0 | −57.3% |
| Sulfate | 235.0 | 194.0 | −17.4% |
| pH, ppm | 7.12 | 7.63 | +06.8% |
| Standard Plate Count, | 510,000 CFU/mi | 18,300 CFU/mi | −96.5% |
| ppm | | | |

The plate count of the pond water was particularly high because the collected water had been standing for 120 days before processing. The post treated water also included the addition of a like volume of city feed water. A 96.5% reduction in plate count was accomplished. The expected reduction in minerals content by 50% did not occur because the city water added to the treated water apparently also contained a certain quantity of these dissolved materials. For example, there was a 57% reduction in the nitrates concentration, which suggests that the city water mixed with the treated water may have also included some nitrates. For comparison purposes, with a typical runoff from a nursery having a plate count of about 200,000, the post-treated water had a plate count of 387, for a reduction of 99.8%.

In a simplified embodiment, as shown in FIG. 6, the water collected as runoff 10 from the field is fed by a pump 34 through a series of filters 136 to remove particulates and carbon filters 138 to remove some of the organic contaminants. The water is then exposed to U.V. light 140 followed by injection of ozone from an ozone generator 142 into the water steam. The water stream is then fed into a contact tank 22 where the treated water is held for a period of time sufficient for the dissolved ozone to react with the organic contaminants in the water. The water can then be passed through a final set of particulate filters 136 and returned to the field. Sensors 38, typically pH, bioburden, oxydation reduction potential (ORP), electronic conductivity, flow rate and temperature are shown to monitor the quality of the water being returned to the field.

Table 2 lists the cost of water for several California communities and the potential savings if runoff water can be collected, treated and recycled for agricultural use at from 25 to 100 gallons per minute.

TABLE 2
CALIFORNIA AGRICULTURAL WATER COSTS
AND POTENTIAL SAVINGS

| | Cost per | Potential Savings | | | |
|---|---|---|---|---|---|
| District | Acre Ft. | 25 gpm | 50 gpm | 75 gpm | 100 gpm |
| San Dieguito | $640.47 | $25,490 | $50,917 | $76,408 | $101,899 |
| San Diego | $583.84 | $23,237 | $46,415 | $69,652 | $92,889 |
| Vista | $522.83 | $20,809 | $41,565 | $62,374 | $83,182 |
| Oceanside | $525.26 | $20,905 | $41,758 | $62,664 | $83,569 |
| Carlsbad | $562.05 | $22,370 | $44,683 | $67,053 | $89,422 |
| Escondido | $436.71 | $17,381 | $34,718 | $52,100 | $69,481 |
| Fallbrook | $423.67 | $16,862 | $33,682 | $50,544 | $67,406 |
| Olivenhain | $811.58 | $32,301 | $64,521 | $96,821 | $129,122 |
| Vallecitos | $527.20 | $20,983 | $41,912 | $62,895 | $83,878 |
| Carpinteria | $604.35 | $24,053 | $48,046 | $72,099 | $96,152 |
| Santa Barbara | (step up) | $67,561 | $137,345 | $207,130 | $276,914 |
| Goleta | $402.90 | $16,022 | $32,044 | $48,066 | $64,088 |

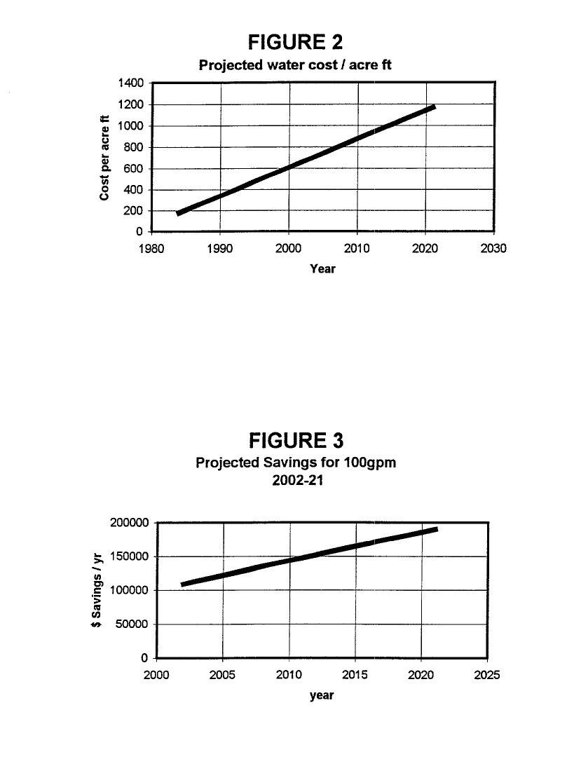

Based on previous, current and projected sources and previous and current costs for a selected water district (Carpenteria Water District, Carpenteria, Calif.) the projected water costs through the year 2030, shown in FIG. 2, can be expected to increase significantly. A 100 gallon/min above described reclamation and recycling system for agricultural water in that Water District, as shown in FIG. 3, is projected to generate a savings, over 20 years, in the cost of water alone of about $3 million.

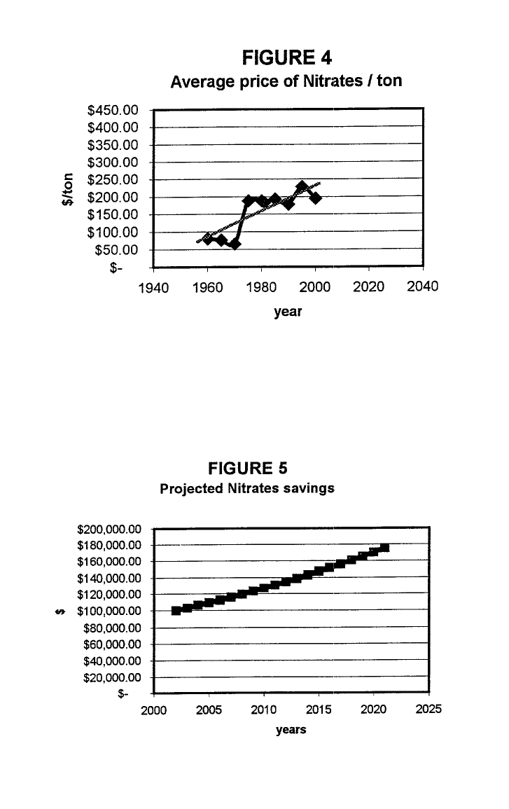

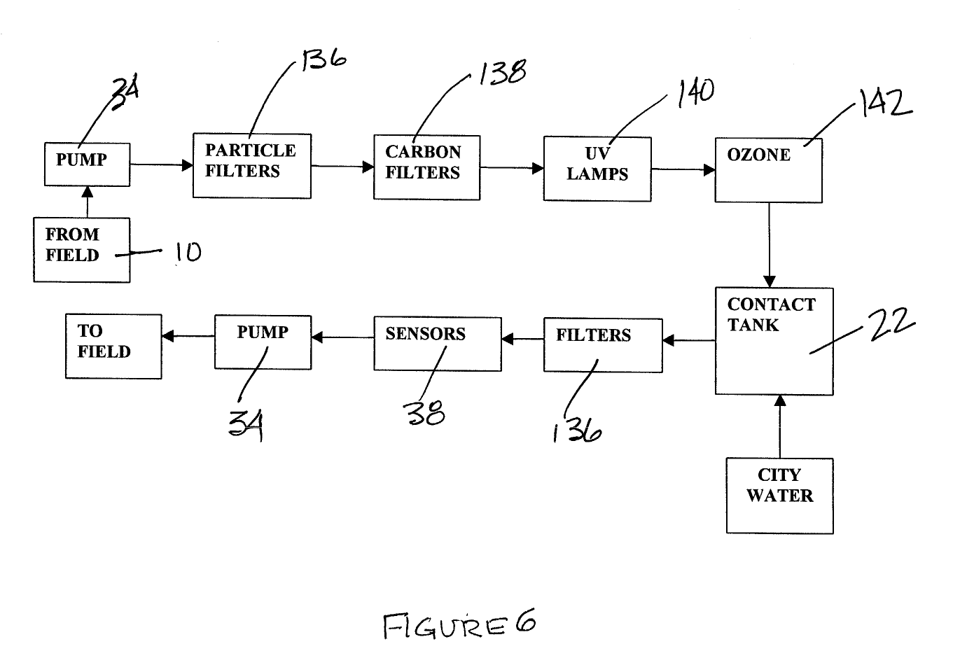

FIG. 4 shows the historical average cost fir 9 different nitrates taken from data published by the USDA for 1960-2000. While about 80% of the fertilizer used can be recovered by recycling the runoff water, based on reutilization of only 50% of the fertilizer the cost savings on fertilizer as a result of recycling is substantial. For the same 100 gallon/min. recycling system, the total costs saving for nitrogen based fertilizers recycled with the water, as shown in FIG. 5, is approximately $2.7 million.

In addition, the crops appear to be healthier. Pesticide demand is typically determined by nonscientific methods. For example, the grower will examine plants for growing deficiencies and the presence of pests. In early growth stages in flowering plants when Botrytis (clear spots on flower petals) or fungus in the pollen caps is observed a pesticide is called for. In standard plants, another method is to use monitoring devices placed near the boundary of planter areas, such as sticky tape or bug traps. In later growth stages, plant damage, such as chewed leaves, bulb holes, leaf curl or trails on leaves are watched for. Still further, the observation of insects on plants will trigger the application of pesticides. However, such treatments have only limited success.

As an example, spider mites, which chew the backside of leave, are one of the most common insects that infest nursery plants. In these instances, treatment must be applied quickly so the mites can be eradicated in less then a week. Spider mites can lay 80,000 eggs/day. These eggs have only a 4 day incubation period. A typical treatment is to a spider miteside. An 8 oz boffle of spider miteside ($350/bottle), which is suggested to treat about 10000 sq ft of leaf surface, will not kill eggs. Failure to completely eliminate the infestation will only require repetitive treatments which will also have limited success.

Based on the uses of such plant inspections, using reclaimed water which has been treated with ozone using the system described above has been found to reduced pesticide requirements by about 20%. This is probably due to the residual ozone in the water which disrupts the normal insect breeding cycle, thus reducing the level of infestation.

As an added benefit, reclamation and recycle of the agricultural water reduces the likelihood of fines for violating runoff clean water standards which can be as much as $3000 per day. Many programs under local and federal clean water regulations also provide for a 50% reduction in fines if that portion of the fine is applied to a remeadiation program for prevention of contaminated runoff.

We claim:

1. A process for reclaiming and recycling agricultural water comprising collecting runoff water from a crop field, treating the runoff water with ozone and returning the ozone treated water to the field within about 1 hour of adding ozone to the runoff water.

2. The process of claim 1 further including exposing the runoff water to high intensity ultraviolet light.

3. The process of claim 2 further including exposing the runoff water to carbon filtration prior to exposure to high intensity ultraviolet light or ozone.

4. The process of claim 2 wherein the recycling of each acre-foot of water reduces the nitrogen fertilizer requirements for a crop by up to about 6000 pounds

5. The process of claim 2 wherein from about 50% to about 80% by weight of nitrogen based fertilizer is recovered and recycled.

6. The process of claim 5 wherein the pesticide requirements for a crop is reduced by up to about 20%.

7. The process of claim 2 wherein, for a crop., nitrogen fertilizer requirements are reduced by from about 50% to about 80%, the pesticide requirements are reduced up to about 20% and about 50% of the water applied to the crop is recovered and reapplied to the crop.

8. A process for improving the quality of agricultural crops, reducing the requirements for nitrogen based fertilizers, pesticides and herbicides, and reducing the cost of water required to irrigate crops comprising collecting runoff water from a crop field, treating the runoff water with ozone and returning the ozone treated water to the field within about 1 hour of adding ozone to the runoff water.

9. The process of claim 8 further including exposing the runoff water to high intensity ultraviolet light.

10. The process of claim 9 further including exposing the runoff water to carbon filtration prior to exposure to high intensity ultraviolet light or ozone.

11. A process for improving the quality of agricultural crops, reducing the requirements for nitrogen based fertilizers by from about 50% to about 80%, reducing the requirements for pesticides and herbicides by up to about 20%, and reducing the cost of water required to irrigate crops by up to about 50% comprising collecting runoff water from a crop field, treating the runoff water with ozone and returning the ozone treated water to the field.

12. The process of claim 12 wherein the treated water is returned to the crop to be treated within about 1 hour of adding ozone to the runoff water.

13. The process of claim 12 wherein the runoff water is exposed to high intensity UV light prior to exposure to ozone.

14. The process of claim 12 wherein the runoff water is passed through a carbon filter before it is exposed to the high intensity UV light.
